(19) United States
(12) Patent Application Publication
(10) Pub. No.: US 2012/0310217 A1
(43) Pub. Date: Dec. 6, 2012

(54) GUIDEWIRE

(75) Inventor: Hideaki MAKI, Nagoya-shi (JP)

(73) Assignee: ASAHI INTECC CO., LTD., Nagoya-shi (JP)

(21) Appl. No.: 13/474,153

(22) Filed: May 17, 2012

(30) Foreign Application Priority Data
Jun. 6, 2011 (JP) 2011-126468

(51) Int. Cl. A61M 25/09

(52) U.S. Cl. 604/528

(57) ABSTRACT

A guidewire includes a core shaft, a coil body, and a tip portion. The coil body covers a distal end portion of the core shaft. The tip portion is formed by coupling a distal end of the coil body and a distal end of the core shaft using metal solder. The tip portion includes an outside diameter decreasing portion and a hemisphere-shaped portion. The outside diameter of the outside diameter decreasing portion decreases linearly in a direction of a distal end. The hemisphere-shaped portion is positioned at a distal end of the outside diameter decreasing portion.

### CROSS-REFERENCE TO RELATED APPLICATION

This application claims priority to Japanese Patent Application No. 2011-126468 filed in the Japan Patent Office on Jun. 6, 2011, the entire contents of which are hereby incorporated by reference.

### BACKGROUND

The disclosed embodiments relate to a medical device. More specifically, the disclosed embodiments relate to a guidewire. Hitherto, various guidewires used for guiding, for example, medical devices to target parts by being inserted into body tissues or tubes such as blood vessels, alimentary canals, or ureters, have been proposed. In recent years, many internal treatments using guidewires have been performed on vascularly occluded lesions that cause ischemic diseases. It is known that microchannels exist in the vascularly occluded lesions. Guidewires that are currently being developed are required to have increased insertability and passability properties with respect to the microchannels in the vascularly occluded lesions.

For example, the guidewire discussed in U.S. Patent Application Publication No. 2007/0185415 (Patent Document 1) includes a distal tip portion formed by a weld at a distal end of the guidewire. The distal tip portion is formed with decreasing outside diameter towards a distal end or in the form of a chisel, to increase the insertability into vascularly occluded lesions.

The guidewire discussed in Japanese Unexamined Patent Application Publication 2007-089901 has a protrusion at a distal end of a distal tip portion, to increase the insertability into vascularly occluded lesions.

### SUMMARY

In the guidewire discussed in Patent Document 1, since a distal tip portion is formed by a weld, it is necessary to melt the material of a coil and the material of a core in the vicinity of the distal tip portion. Therefore, heat used during welding causes the core and the coil to be in an annealed state, thereby considerably reducing mechanical strength. When the core and the coil are formed of different types of metals, the melting point of the core and the melting point of the coil differ from each other. Therefore, sufficient weld strength cannot be obtained, as a result of which mechanical strength of the distal tip portion is further reduced. Consequently, when the guidewire discussed in Patent Document 1 is inserted into vascularly occluded lesions, a distal end portion of the guidewire is deformed, as a result of which the guidewire does not have sufficient insertability into the vascularly occluded lesions.

Further, it is difficult to form the distal tip portion with a spherical shape. Therefore, it takes a lot of time to taper the distal tip portion after welding.

The guidewire discussed in Patent Document 2 has increased insertability into vascularly occluded lesions due to the protrusion formed at the distal end of the distal tip portion. However, since the outside diameter increases suddenly from a proximal end of the protrusion towards the distal end of the distal tip portion, the resistance of the guidewire against the vascularly occluded lesions is high. As a result, the guidewire discussed in Patent Document 2 does not have sufficient passability with respect to vascularly occluded lesions.

Accordingly, in view of such problems, it is an object of the present invention to provide a guidewire that has excellent insertability and passability with respect to vascularly occluded lesions, and a guidewire that makes it possible to achieve high productivity.

According to an aspect of the present invention, there is provided a guidewire including a core shaft, a coil body that covers the core shaft, and a tip portion where a distal end of the coil body and a distal end of the core shaft are joined using metal solder. The tip portion includes an outside diameter decreasing portion where an outside diameter of the tip portion decreases in a direction of a distal end of the tip portion, and a hemisphere-shaped portion provided at a distal end of the outside diameter decreasing portion. An outside diameter of a proximal end of the hemisphere-shaped portion is the same as an outside diameter of the distal end of the outside diameter decreasing portion.

Since the guidewire according to the aspect of the present invention is formed using metal solder, it is possible to reduce the influence of heat on the core shaft and the coil to prevent the mechanical strength of the distal end of the guidewire from being reduced, so that, even if the guidewire is inserted into vascularly occluded lesions, it is possible to prevent deformation of the distal end portion of the guidewire, thereby making it is possible to increase the insertability of the guidewire into the vascularly occluded lesions. In addition, since metal solder is used, it possible to easily form the tip portion at the distal end of the guidewire, and to achieve excellent productivity. Further, since the distal end of the distal tip portion is provided with a hemisphere-shaped portion whose outside diameter is smaller than that of the distal tip portion, it is possible to move the guidewire to microchannels in the vascularly occluded lesions. Still further, since, when extending from the outside-diameter decreasing portion to the hemisphere-shaped portion, the outside diameter is constant, it is possible to prevent the guidewire from being caught in the vascularly occluded lesions, and to increase the insertability and the passability of the guidewire with respect to the vascularly occluded lesions.

### BRIEF DESCRIPTION OF THE DRAWINGS

FIGS. 1A to 1E illustrate structures of a guidewire according to an embodiment of the present invention, with FIG. 1A illustrating the entire guidewire, FIG. 1B being an enlarged view of a tip portion illustrated in FIG. 1A, FIG. 1C illustrating the tip portion as viewed from a distal end of the guidewire towards a proximal end of the guidewire, and FIGS. 1D and 1E illustrating modifications of the embodiment and being enlarged views of the tip portion.

FIG. 2 illustrates an entire guidewire according to an embodiment of the present invention.

FIGS. 3A to 3D illustrate structures of a guidewire according to an embodiment of the present invention, with FIG. 3A illustrating the entire guidewire, FIG. 3B being an enlarged view of a tip portion illustrated in FIG. 3A, and FIGS. 3C and 3D illustrating modifications of the embodiment and being enlarged views of the tip portion.

FIGS. 4A and 4B illustrate a structure of a guidewire according to an embodiment of the present invention, with FIG. 4A illustrating the entire guidewire and FIG. 4B illustrating a modification of the embodiment and being an enlarged view of a tip portion.

### DETAILED DESCRIPTION OF EMBODIMENTS

Guidewires according to embodiments of the present invention will now be described on the basis of preferred embodiments shown in the drawings.

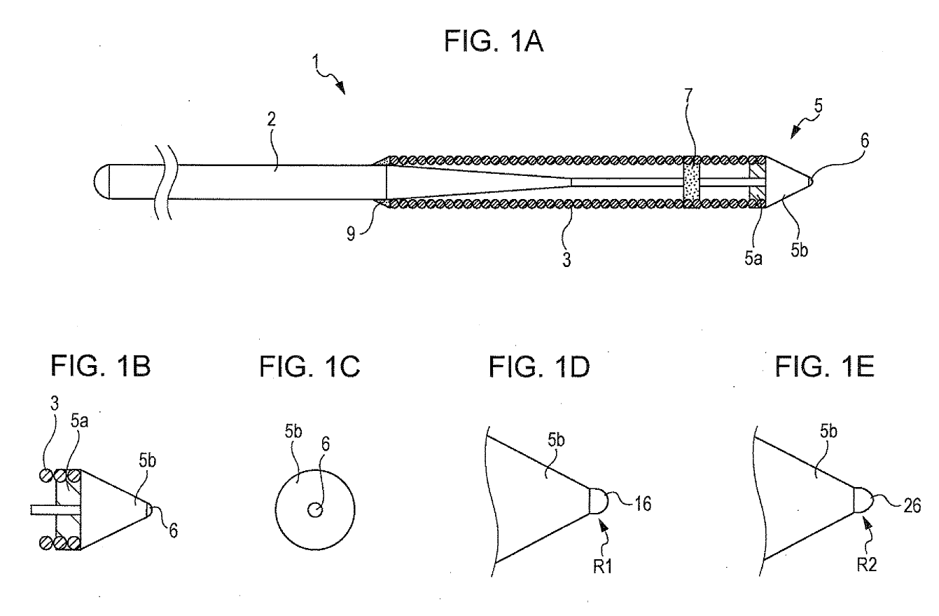

FIGS. 1A to 1E illustrate a guidewire 1 according to an embodiment of the present invention.

In FIGS. 1A to 1E, for convenience of explanation, the left side corresponds to a proximal end and the right side corresponds to a distal end.

In FIG. 1A, for facilitating understanding, a lengthwise direction of the guidewire 1 is reduced. In addition, since FIG. 1A schematically illustrates the entire guidewire 1, the dimensions of the entire guidewire 1 differ from the actual dimensions thereof.

In FIG. 1A, the guidewire 1 includes a core shaft 2, a coil body 3 that covers a distal end of the core shaft 2, and a tip portion 5 where a distal end of the coil body 3 and the distal end of the core shaft 2 are joined. In the direction of a proximal end of the tip portion 5, the coil body 3 and the core shaft 2 are joined by an intermediate joining portion 7, and a proximal end of the coil body 3 and the core shaft 2 are joined by a proximal-end joining portion 9.

The tip portion 5 includes a proximal-end tip portion 5a, an outside diameter decreasing portion 5b, and a hemisphere-shaped portion 6. The distal end of the coil body 3 and the distal end of the core shaft 2 are joined at the proximal-end tip portion 5a. The outside diameter of the outside diameter decreasing portion 5b decreases linearly in the direction of the distal end. The hemisphere-shaped portion 6 is provided at a distal end of the outside diameter decreasing portion 5b.

In FIG. 1B, a boundary portion between the distal end of the outside diameter decreasing portion 5b and a proximal end of the hemisphere-shaped portion 6 has a constant outside diameter. Accordingly, the guidewire 1 is formed so that its state smoothly changes from a state in which the outside diameter of the outside diameter decreasing portion 5b gradually decreases linearly, and, then, to a state in which the outside diameter of the hemisphere-shaped portion 6 decreases so as to form a curve.

When the guidewire 1 shown in FIG. 1B is viewed from the distal end of the guidewire 1 towards the proximal end of the guidewire 1, the outside diameter decreasing portion 5b and the hemisphere-shaped portion 6 have circular shapes that are not distorted in a particular way in a particular direction as shown in FIG. 1C.

Accordingly, the tip portion 5 of the guidewire 1 has the outside diameter decreasing portion 5b whose outside diameter decreases linearly towards the distal end, and the hemisphere-shaped portion 6 provided at the distal end of the outside diameter decreasing portion 5b. In addition, the outside diameter at the boundary portion between the distal end of the outside diameter decreasing portion 5b and the proximal end of the hemisphere-shaped portion 6 is constant. Therefore, it becomes easier to insert the hemisphere-shaped portion 6 into an entrance of a microchannel in a vascularly occluded lesion, and the guidewire 1 can be easily passed through the interior of the vascularly occluded lesion without receiving a high resistance even when the guidewire 1 passes through the interior of the vascularly occluded lesion. The form of the guidewire 1 makes it possible to increase the insertability and passability of the guidewire 1 with respect to the interior of the vascularly occluded lesion.

Since the outside diameter of the outside diameter decreasing portion Sb decreases linearly towards the distal end, it is possible to further reduce the resistance applied to the guidewire 1 when the guidewire 1 is inserted into the vascularly occluded lesion. Therefore, it is possible to further increase the insertability of the guidewire 1 into the vascularly occluded lesion.

The outside diameter decreasing portion Sb and the hemisphere-shaped portion 6 have circular shapes that are not distorted in a particular way in a particular direction. Therefore, when the hemisphere-shaped portion 6 is inserted into a microchannel in a vascularly occluded lesion, it is possible to prevent the hemisphere-shaped portion 6 from being caught by an entrance of the microchannel. Consequently, it is possible to insert the guidewire 1 into the microchannel in the vascularly occluded lesion, and the guidewire 1 is not often subjected to resistance in the interior of the vascularly occluded lesion even when the guidewire 1 is pushed into and passed through the interior of the vascularly occluded lesion or passed through the interior of the vascularly occluded lesion while rotating the guidewire 1. Thus, by forming the outside diameter decreasing portion 5b and the hemisphere-shaped portion 6 with such circular shapes, it is possible to further increase the insertability and the passability of the guidewire 1 with respect to the interior of the vascularly occluded lesion.

FIG. 1D illustrates a modification of the embodiment. Comparing a hemisphere-shaped portion 16 with the hemisphere-shaped portion 6 illustrated in FIG. 1A, the hemisphere-shaped portion 16 has a completely hemispherical shape (that is, has a shape that is half a true circle). By forming the hemisphere-shaped portion 16 with a completely hemispherical shape, the hemisphere-shaped portion 16 protrudes in the direction of the distal end of the guidewire 1. At a boundary portion between the distal end of the outside diameter decreasing portion 5b and the proximal end of the hemisphere-shaped portion 16, the distal end of the outside diameter decreasing portion 5b and a proximal end of the hemisphere-shaped portion 16 are connected to each other so as to form a curve R1.

Accordingly, when the hemisphere-shaped portion 16 having a completely hemispherical shape is provided on the guidewire 1, the hemisphere-shaped portion 16 protrudes in the direction of the distal end of the guidewire 1. As a result, it becomes easier to move the guidewire 1 to a microchannel in a vascularly occluded lesion, thereby making it possible to further increase the insertability of the guidewire 1 into the vascularly occluded lesion.

Further, since, at the boundary portion between the distal end of the outside diameter decreasing portion 5b and the proximal end of the hemisphere-shaped portion 16, the distal end of the outside diameter decreasing portion 5b and the proximal end of the hemisphere-shaped portion 16 are connected to each other so as to form the curve R1, it is possible to further reduce resistance applied to the boundary portion when the boundary portion passes through the vascularly occluded lesion. Consequently, it is possible to further increase passability of the guidewire 1 with respect to the vascularly occluded lesion.

FIG. 1E also shows a modification of the embodiment. A hemisphere-shaped portion 26 has a spindle-like hemispherical shape. Accordingly, comparing the hemisphere-shaped portion 26 with the hemisphere-shaped portion 6 shown in FIG. 1A, the hemisphere-shaped portion 26 protrudes by a greater amount in the direction of the distal end of the guidewire 1. At a boundary portion between the distal end of the outside diameter decreasing portion 5b and a proximal end of the hemisphere-shaped portion 26, the distal end of the outside diameter decreasing portion 5b and the proximal end of the hemisphere-shaped portion 26 are connected to each other so as to form a curve R2.

Accordingly, since the hemisphere-shaped portion 26 has a spindle-like shape, the hemisphere-shaped portion 26 protrudes by a greater amount in the direction of the distal end of the guidewire 1, making it easier to move the guidewire 1 to a microchannel of a vascularly occluded lesion. Therefore, it is possible to further increase the insertability of the guidewire 1 into the vascularly occluded lesion. Additionally, at the boundary portion between the distal end of the outside diameter decreasing portion 5b and the proximal end of the hemisphere-shaped portion 26, the distal end of the outside diameter decreasing portion 5b and the proximal end of the hemisphere-shaped portion 26 are connected to each other so as to form the curve R2. This makes it possible for the boundary portion between the distal end of the outside diameter decreasing portion 5b and the proximal end of the hemisphere-shaped portion 26 to further reduce the resistance applied to the boundary portion when the boundary portion passes through the interior of the vascularly occluded lesion. Therefore, it is possible to further increase the passability of the guidewire 1 with respect to the vascularly occluded lesion.

Next, the materials of the structural parts in the embodiment will be described. Although the usable materials of the core shaft 2 are not particularly limited, materials such as stainless steel (such as SUS304), superelastic alloys (such as Ni—Ti alloys), and piano wires may be used.

Examples of the materials of the tip portion 5, the intermediate joining portion 7, and the proximal-end joining portion 9 for joining the core shaft 2 and the coil body 3 include metal solders, such as Sn—Pb solders, Pb—Ag solders, Sn—Ag solders, and Au—Sn solders. In order to minimize a reduction in mechanical strength caused by the influence of heat on the core shaft 2 and on the coil body 3, it is desirable to use metal solders having melting points that are lower than those of the materials of the coil body 3 and the materials of the core shaft 2 such as those mentioned above. Further, in order to reliably prevent a reduction in mechanical strength caused by the influence of heat on the core shaft 2 and on the coil body 3, it is desirable to use metal solders having melting points that are equal to or less than 500 degrees.

In particular, it is desirable that, for the tip portion 5, metal solders such as Au—Sn solders whose principal component is gold be used. Metal solders, such as Au—Sn solders, are known to have high rigidity. When the tip portion 5 is formed of an Au—Sn alloy, the tip portion 5 can have the proper rigidity, thereby increasing the insertability of the guidewire 1 into a vascularly occluded lesion. The melting point of Au—Sn alloy is equal to or less than 400 degrees, so that the Au—Sn alloy metal solder makes it possible to prevent a reduction in mechanical strength caused by the influence of heat on the core shaft 2 and on the coil body 3.

Since metal solders, such as Au—Sn solders, are highly impermeable to radiation, such solders can increase visibility for a radiation transparent image at the tip portion 5 of the guidewire 1. As a result, it is possible to operate the guidewire 1 while knowing the position of the guidewire 1 in a vascularly occluded lesion with a high degree of certainty. This is advantageous when the guidewire 1 is being passed through the interior of the vascularly occluded lesion.

When assembling the core shaft 2 and the coil body 3 using metal solder, it is desirable to previously apply flux to a portion where the core shaft 2 and the coil body 3 are to be joined. This results in good wettability of the metal solder with respect to the core shaft 2 and the coil body 3, and to increase bonding strength.

By forming the tip portion 5 of the guidewire 1 using metal solder in this way, it is possible to suppress a reduction in mechanical strength caused by the influence of heat on the coil body 3 and the core shaft 2 of the guidewire 1. This makes it possible to ensure that the mechanical strength of a distal end portion of the guidewire 1 has not been compromised during manufacturing. Consequently, it is possible to increase the insertability of the guidewire 1 into a vascularly occluded lesion. In addition, as described below, metal solders can be used to easily form the tip portion 5 by using, for example, a soldering iron, so that it is possible to achieve excellent productivity when forming the tip portion 5 of the guidewire 1.

Usable materials of the coil body 3 include wires that are impermeable to radiation and wires that are permeable to radiation.

Usable materials of the wires that are impermeable to radiation are not particularly limited, and include gold, platinum, tungsten, and alloys of these elements (such as platinum-nickel alloys).

Usable materials of the wires that are permeable to radiation are not particularly limited, and include stainless steel (SUS304, SUS316, etc.), superelastic alloys (such as Ni—Ti alloys), and piano wires.

The guidewire 1 according to the embodiment may be manufactured by the following method.

First, an outer periphery of one end of a metal wire is ground by a centerless grinding machine, to manufacture a core shaft 2 whose distal end portion has a smaller outside diameter.

Next, coil wires are wound around a cored bar for a coil, and the coil wires are subjected to heat treatment while they are wound around the cored bar, after which the cored bar is removed, thereby manufacturing a coil body 3.

Then, a distal end of the core shaft 2 is inserted from a proximal end of the coil body 3, and the proximal end of the coil body 3 and the core shaft 2 are joined together with metal solder using, for example, a soldering iron, to form a proximal-end joining portion 9.

Next, using, for example, the soldering iron, a distal end of the coil body 3 and the distal end of the core shaft 2 are joined using metal solder. At this time, a proximal-end tip portion 5a and a previous form portion are formed. The proximal-end tip portion 5a is joined to the distal end of the core shaft 2 when metal solder enters the interior of the coil body 3. The previous form portion has an overall hemispherical shape and is formed at a distal end side of the proximal-end tip portion 5a.

Next, in the direction of a proximal end of the proximal-end tip portion 5a, the core shaft 2 and the coil body 3 are joined using metal solder, to form an intermediate joining portion 7.

Lastly, using a device, such as Leutor, the previous form portion is ground, to form an outside diameter decreasing portion 5b and a hemisphere-shaped portion 6.

From the viewpoint of strength, it is desirable to integrally form the outside diameter decreasing portion 5b and the hemisphere-shaped portion 6. However, after forming the previous form portion of a hemisphere-shaped portion 6 using, for example, metal solder after forming the outside diameter decreasing portion 5b, it is possible to grind the previous form portion again, and adjust its shape using, for example, Leutor. When, for example, metal solder is further used after forming the outside diameter decreasing portion 5b, it is possible to use a different type of metal solder. However, considering the welding temperature and the joining strength with the outside diameter decreasing portion 5b, it is desirable to use the same type of metal solder.

The manufacturing method of the guidewire 1 is not limited to this manufacturing method, so that the tip portion 5 may be manufactured by other methods. For example, it is possible to, by using a die whose hollow portion has the shape of the outside diameter decreasing portion 5b and the shape of the hemisphere-shaped portion 6, set the distal end of the core shaft 2 and the distal end of the coil body 3, and pour molten metal solder into the die, to form the tip portion 5.

Although, in the embodiment, the distal end of the core shaft 2 is disposed in the proximal-end tip portion 5a, the present invention is not limited thereto. It is possible to dispose the distal end of the core shaft 2 in the outside diameter decreasing portion 5b or the hemisphere-shaped portion 6. When the distal end of the core shaft 2 is disposed in the outside diameter decreasing portion 5b or the hemisphere-shaped portion 6, it is possible to increase the push-in characteristic of the guidewire 1. Therefore, it is possible to increase the insertability and passability of the guidewire 1 with respect to a vascularly occluded lesion.

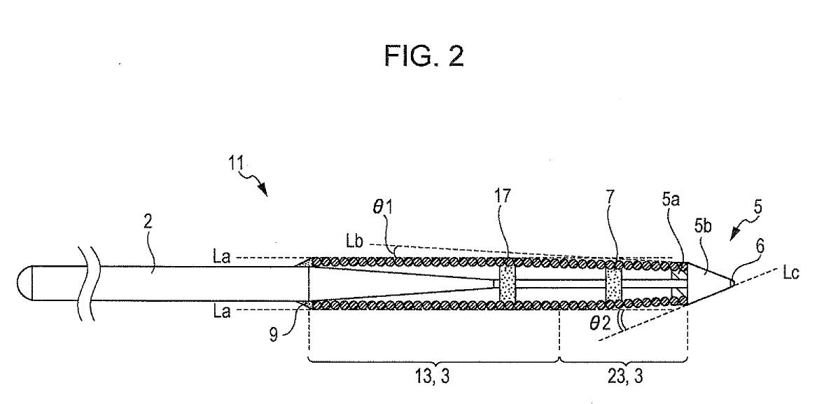

Next, a guidewire 11 according to an embodiment will be described with reference to FIG. 2 by focusing on the differences between the guidewire 11 according to the embodiment and the guidewire 1 according to the above-discussed embodiment. Portions that correspond to those in the above-discussed embodiment will be given the same reference numerals in FIG. 2.

In FIG. 2, for facilitating understanding, a lengthwise direction of the guidewire 11 is reduced, and the entire guidewire 11 is schematically illustrated, so that the dimensions of the entire guidewire 11 differ from the actual dimensions thereof.

In FIG. 2, the guidewire 11 according to the embodiment has the same structural features as the guidewire 1 according to the above-discussed embodiment except that the guidewire 11 includes a constant outside diameter portion 13, a tapered portion 23, and an intermediate joining portion 7. The constant outside diameter portion 13 is where a proximal end side of a coil body 3 has a constant coil outside diameter. The tapered portion 23 is where a distal end side of the coil body 3 has a coil outside diameter that decreases in a direction from the proximal end side of a coil body 3 toward of a distal end thereof. The second intermediate joining portion 17 is where a core shaft 2 and the coil body 3 are joined in a direction of a proximal end of the second intermediate joining portion 17.

Here, in the coil body 3 shown in FIG. 2, a tangent line that contacts a coil outer periphery at the constant outside diameter portion 13 is defined as a straight line La, a tangent line that contacts the coil outer periphery at the tapered portion 23 is defined as a straight line Lb, and, in a tip portion 5 shown in FIG. 2, a tangent line that contacts an outer periphery of an outside diameter decreasing portion 5b is defined as a straight line Lc. In this case, an angle formed between the straight lines La and Lb is angle theta 1 (θ1). The angle θ1 corresponds to a taper angle of the tapered portion 23 with respect to a center axis of the guidewire 11 in a long-axis direction of the guidewire 11. The angle formed between the straight lines La and Lc is angle theta 2 (θ2). The angle θ2 is an angle by which the outside diameter of the outside diameter decreasing portion 5b decreases with respect to the center axis of the guidewire 11 in the long-axis direction of the guidewire 11.

In the embodiment, the angle θ2 by which the outside diameter of the outside diameter decreasing portion 5b decreases is set so as to be larger than the angle θ1 of the tapered portion 23 so that the decreasing degree (i.e., the degree of tapering) of the outside diameter of the outside diameter decreasing portion 5b is greater than the decreasing degree of the outside diameter of the tapered portion 23. That is, the decreasing degree, or degree of tapering is the amount of tapering, as measured by an angle formed by the intersection of an imaginary line passing through the central axis of the guidewire and either: i) a line passing through two points on the outside diameter decreasing portion Sb (i.e., measured by angle θ2), or ii) a line passing through two points on the coil outer periphery at the tapered portion 23 (i.e., measured by angle θ1).

Accordingly, in the guidewire 11 according to the embodiment, the decreasing degree of the outside diameter of the outside diameter decreasing portion 5b is greater than the decreasing degree of the outside diameter of the tapered portion 23. Therefore, at a boundary portion between a proximal end of the outside diameter decreasing portion 5b and a distal end of the tapered portion 23, it is possible to reduce resistance in a vascularly occluded lesion when the guidewire 11 passes through the vascularly occluded lesion. This makes it possible to increase passability of the guidewire 11 with respect to the vascularly occluded lesion.

The coil body 3 including the constant outside diameter portion 13 and the tapered portion 23 can be manufactured by providing, at a cored bar for a coil, a portion whose outside diameter is constant and a portion whose outside diameter decreases, and by using the cored bar when forming the coil body 3.

In the guidewire 11 according to the embodiment, the decreasing degree of the outside diameter of the outside diameter decreasing portion 5b may be any degree as long as it is greater than the decreasing degree of the outside diameter of the tapered portion 23. For example, the outside diameter of the tapered portion 23 of the coil body 3 and the outside diameter of the outside diameter decreasing portion 5b may decrease so as to form an asymptotic curve.

The second intermediate joining portion 17 may be formed of the same material as the intermediate joining portion 7. The guidewire 11 according to the embodiment includes the second intermediate joining portion 17 where the constant outside diameter portion 13 of the coil body 3 and the core shaft 2 are joined, and the intermediate joining portion 7 where the tapered portion 23 and the core shaft 2 are joined. This makes it possible to join each portion of the coil body 3 and the core shaft 2. Therefore, the insertability and passability of the guidewire 11 with respect to a vascularly occluded lesion can be increased.

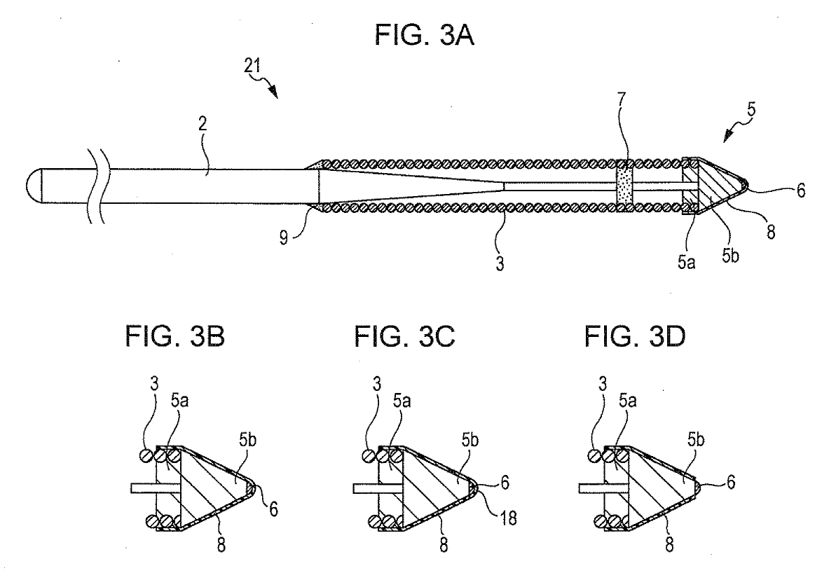

Next, using FIGS. 3A and 3B, a guidewire 21 according to an embodiment will be described by focusing on the differences between the guidewire 21 according to the embodiment and the guidewire 1 according to the above-discussed embodiment. Portions that correspond to those in the above-discussed embodiment will be given the same reference numerals in FIGS. 3A and 313.

In FIG. 3A, for facilitating understanding, a lengthwise direction of the guidewire 21 is reduced, and the entire guidewire 21 is schematically illustrated, so that the dimensions of the entire guidewire 21 differ from the actual dimensions thereof. FIG. 3B is an enlarged view of a tip portion 5 shown in FIG. 3A.

In FIGS. 3A and 3B, the guidewire 21 has the same structural features as the guidewire 1 according to the above-discussed embodiment except that a lubricant coating 8 is applied to an outer periphery of the tip portion 5. In the embodiment, from FIG. 3B, the lubricant coating 8 covers an outer periphery of a proximal-side tip portion 5a, an outer periphery of an outside diameter decreasing portion 5b, and an outer periphery of a hemisphere-shaped portion 6.

Since, in the guidewire 21 according to the embodiment, the lubricant coating 8 is applied to the outer periphery of the tip portion 5, it is possible to increase the passability of the guidewire 21 with respect to a vascularly occluded lesion.

Usable materials for the lubricant coating 8 are not particularly limited, and include hydrophobic coating materials, such as fluorocarbon resin and silicone oil, and hydrophilic coating materials, such as polyvinyl pyrrolidone, polyacrylic acid, polyacrylamide, polyvinyl alcohol, maleic anhydride copolymer, and hyaluronic acid. In order to increase the passability of the guidewire 21 with respect to a vascularly occluded lesion, it is desirable that the lubricant coating 8 be formed of a hydrophilic coating material.

FIG. 3C illustrates a modification of the embodiment. In FIG. 3C, the lubricant coating 8 is applied to the outer periphery of the proximal-end tip portion 5a of the tip portion 5 and the outer periphery of the outside diameter decreasing portion 5b of the tip portion 5, and a low-lubricity coating 18 having a friction resistance with respect to biological tissue that is higher than that of the lubricant coating is applied to the outer periphery of the hemisphere-shaped portion 6 of the tip portion 5.

Accordingly, when the lubricant coating 8 is applied to the outer periphery of the proximal-end tip portion and the outer periphery of the outside diameter decreasing portion 5b, and when the low-lubricity coating 18 is applied to the outer periphery of the hemisphere-shaped portion 6, the friction resistance of the hemisphere-shaped portion 6 with respect to the biological tissue can be set higher than the friction resistance of the proximal-end tip portion 5a and the outside diameter decreasing portion 5b with respect to the biological tissue. Therefore, when the hemisphere-shaped portion of the guidewire 21 is moved to a microchannel that is situated at an entrance of a vascularly occluded lesion, it is possible to reliably move the hemisphere-shaped portion 6 to the vascularly occluded lesion by preventing the hemisphere-shaped portion 6 from slipping. Consequently, it is possible to significantly increase the insertability of the guidewire 21 with respect to the vascularly occluded lesion. After a distal end of the guidewire 21 (the hemisphere-shaped portion 6) has been inserted into the vascularly occluded lesion, the passability of the guidewire 21 with respect to the vascularly occluded lesion can be increased by the effect of the lubricant coating 8 applied to the outside diameter decreasing portion 5b and the proximal-end tip portion 5a.

If combinations of materials are used for the lubricant coating 8 and the low-lubricity coating 18, for example, when the lubricant coating 8 is formed of a hydrophilic coating agent, the low-lubricity coating 18 is formed of a hydrophobic coating agent. The combinations of materials are not limited thereto, so that resins, such as polyamide resin, polyurethane resin, and various elastomers may be used for the material of the low-lubricity coating 18.

In another modification of the embodiment, as shown in FIG. 3D, it is possible to apply the lubricant coating 8 to the proximal-end tip portion 5a and the outside diameter decreasing portion 5b of the tip portion 5, and not to apply a coating to the hemisphere-shaped portion 6.

In order to coat the guidewire 21 as shown in FIGS. 3C and 3D, it is possible to apply the low-lubricity coating 18 to the hemisphere-shaped portion 6 after applying the lubricant coating 8 to only the proximal-side tip portion 5a and the outside diameter decreasing portion 5b. The method is not limited thereto. Another method may be used. In this method, after the lubricant coating 8 is applied to the entire outer periphery of the tip portion 5, the lubricant coating 8 applied to the hemisphere-shaped portion 6 may be wiped off with a sheet or scraped off by a device such as Leutor. Then, the low-lubricity coating 18 is applied to the hemisphere-shaped portion 6. The sheet is impregnated with a solution, which is a good solvent of the lubricant coating 8.

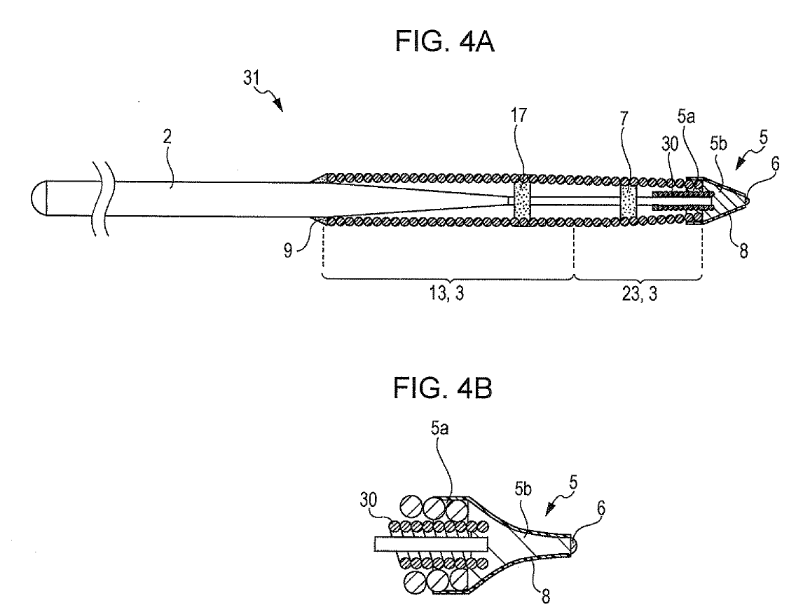

Next with reference to FIG. 4A, a guidewire 31 according to an embodiment will be described by focusing on the differences between the guidewire 31 according to the embodiment and the guidewire 1 according to the above-discussed embodiment. Portions that correspond to those in the above-discussed embodiment will be given the same reference numerals in FIGS. 4A and 4B.

In FIG. 4A, for facilitating understanding, a lengthwise direction of the guidewire 31 is reduced, and the entire guidewire 31 is schematically illustrated, so that the dimensions of the entire guidewire 31 differ from the actual dimensions thereof.

In FIG. 4A, the guidewire 31 has the same structural features as the guidewire 11 according to the above-discussed embodiment except that an inner coil body 30 that covers a distal end portion of a core shaft 2 is provided at an inner side of a distal end portion of a coil body 3, that is, at an inner side of a tapered portion 23, and that a lubricant coating 8 is applied to a portion other than a hemisphere-shaped portion 6 of a tip portion 5.

The inner coil body 30 which is a multi-thread coil body is a formed by twisting a plurality of coil wires. A distal end of the inner coil body 30 is joined along with a distal end of the coil body 3 and a distal end of the core shaft 2 by the tip portion 5. A proximal end of the inner coil body 30 is joined to the core shaft 2 at a position that is closer to a proximal end side than the tip portion 5 and that is closer to a distal end side than an intermediate joining portion 7. The distal end of the inner coil body 30 is disposed closer to the distal end side than the distal end of the coil body 3, and inside an outside diameter decreasing portion 5b of the tip portion 5.

Accordingly, in the guidewire 31 according to the embodiment, the proximal end of the inner coil body 30 formed of a multi-thread coil body is joined to the core shaft 2. While the distal end of the inner coil body 30 is disposed closer to the distal end side than the distal end of the coil body 3, and is positioned in the outside diameter decreasing portion 5b of the tip portion 5, the inner coil body 30 is joined to the distal end of the core shaft 2 and the distal end of the coil body 3 through the tip portion 5. This makes it possible to more easily transmit, for example, a push-in force at a proximal end side of the guidewire 31 to the tip portion 5, so that the insertability of the guidewire 31 into a vascularly occluded lesion is considerably increased.

The inner coil body 30 formed of a multi-thread coil body may be formed of a material that is the same as the material of the coil body 3. However, from the viewpoint of transmitting, for example, a push-in force at the proximal end side of the guidewire 31, it is desirable to use, for example, stainless steel wires having excellent mechanical strength as coil wires of the inner coil body 30.

The method of forming the multi-thread coil body 30 may be the same as the method of forming the coil body 3 except for the winding of a plurality of coil wires around a cored bar.

FIG. 4B illustrates a modification of the embodiment. The outside diameter of the outside diameter decreasing portion 5b of the tip portion 5 shown in FIG. 4B decreases asymptotically. By forming the shape of the outside diameter portion 5b in this way, it is possible to increase the insertability of the guidewire 31 into a vascularly occluded lesion.

Embodiments of the present invention are not limited to the above-described embodiments. Various modifications can be made by those skilled in the art within the technical idea of the present invention.

For example, in order to increase the insertability and passibility of the guidewire 31 with respect to a vascularly occluded lesion, it is desirable to form the inner coil body 30 of the guidewire 31 according to the embodiment shown in FIG. 4 using a multi-thread coil body. However, when the distal end portion of the guidewire 31 is required to have flexibility, a single coil body formed of one coil wire may be used.

The hemisphere-shaped portion 6 of the tip portion 5 whose outside diameter decreases asymptotically as shown in FIG. 4B may be formed as the spindle-like hemisphere-shaped portion 26 shown in FIG. 1E. This makes it possible to further increase the insertability of the guidewire 31 into a vascularly occluded lesion.

While the disclosed embodiments have been shown and described in detail, the foregoing description is in all aspects illustrative and not restrictive. It is therefore understood that numerous modifications and variations can be devised without departing from the spirit and scope of the invention.

What is claimed is:

1. A guidewire comprising:
a core shaft;
a coil body that covers the core shaft; and
a tip portion where a distal end of the coil body and a distal end of the core shaft are coupled by a metal solder, wherein
the tip portion includes: i) an outside diameter decreasing portion where an outside diameter of the tip portion decreases in a direction toward a distal end of the tip portion, and ii) a hemisphere-shaped portion provided at a distal end of the outside diameter decreasing portion, and
an outside diameter of a proximal end of the hemisphere-shaped portion is substantially the same as an outside diameter of the distal end of the outside diameter decreasing portion.

2. The guidewire according to claim 1, wherein
the outside diameter decreasing portion and the hemisphere-shaped portion are connected to each other at a boundary portion, so as to form a curve.

3. The guidewire according to claim 1, wherein
the coil body has a tapered portion where an outside diameter of the coil body decreases in a direction toward the distal end of the coil body, and
a degree of tapering of the outside diameter of the outside diameter decreasing portion is greater than a degree of tapering of the outside diameter of the tapered portion.

4. The guidewire according to claim 1, wherein
the tip portion is coated with a lubricant coating.

5. The guidewire according to claim 4, wherein
the lubricant coating is applied to a portion other than the hemisphere-shaped portion of the tip portion.

6. The guidewire according to claim 4, wherein
in a wet state, a friction resistance of the hemisphere-shaped portion with respect to biological tissue is higher than a friction resistance of the outside diameter decreasing portion with respect to the biological tissue.

7. The guidewire according to claim 1, further comprising
a multi-thread coil body disposed at an inner side of the coil body, the multi-thread coil body covering the core shaft, wherein
a proximal end of the multi-thread coil body is coupled to the core shaft, and
a distal end of the multi-thread coil body is disposed closer to a distal end side than the distal end of the coil body, and is joined to the core shaft in the outside diameter decreasing portion through the tip portion.

8. The guidewire according to claim 2, wherein
a diameter of the hemisphere-shaped portion is greater than a diameter of the boundary portion.

9. The guidewire according to claim 1, wherein
the metal solder has a melting point that is lower than a melting point of the coil body and a melting point of the core shaft.

10. The guidewire according to claim 1, wherein
the core shaft is disposed within at least part of the outside diameter decreasing portion.

11. The guidewire according to claim 1, wherein
the core shaft is disposed within at least part of the hemisphere-shaped portion.
